(19) United States
(12) Patent Application Publication
(10) Pub. No.: US 2026/0363744 A1
(43) Pub. Date: Nov. 19, 2026

(54) METHOD FOR POSITIONING AND GENERATING TITANIUM PLATE FOR INTERNAL FIXATION OF JAWBONE, AS WELL AS DEVICE, MEDIUM, AND PRODUCT

(71) Applicant: Sichuan University, Chengdu City, Sichuan Province (CN)

(72) Inventors: En LUO, Chengdu City, Sichuan Province (CN); Liwei HUANG, Chengdu City, Sichuan Province (CN); Zhaokun ZHU, Chengdu City, Sichuan Province (CN); Yao LIU, Chengdu City, Sichuan Province (CN); Ze HE, Chengdu City, Sichuan Province (CN)

(73) Assignee: Sichuan University, Chengdu City, Sichuan Province (CN)

(21) Appl. No.: 19/644,754

(22) Filed: Apr. 10, 2026

(30) Foreign Application Priority Data
May 16, 2025 (CN) 202510629247.8

(51) Int. Cl. A61B 17/80; A61B 6/50; A61B 17/00; A61B 34/10

(52) U.S. Cl. CPC A61B 17/8071; A61B 34/10; A61B 6/501; A61B 2017/00526; A61B 2034/105; A61B 2034/108

(57) ABSTRACT

A method for positioning and generating titanium plate for internal fixation of jawbone, as well as a device, a medium, and a product are provided. The method includes: performing CT or CBCT scanning on bone surface of patient to determine a bone surface digital model; performing surface smoothing on mesh data to determine each mesh point; determining a normal vector of triangular patch based on an adjacent triangular patch, and determining bone surface thickness value of each mesh point; mapping bone surface thickness value of each mesh point to the bone surface digital model to generate thickness map, identifying mesh region corresponding to maximum bone surface thickness value, and determining a target triangular patch corresponding to the mesh region; and generating positioning of each titanium plate screw hole; and generating a titanium plate for internal fixation of jawbone by using bone surface mesh patch offset method.

### CROSS-REFERENCE TO RELATED APPLICATION

This patent application claims the benefit and priority of Chinese Patent Application No. 202510629247.8 filed with the China National Intellectual Property Administration on May 16, 2025, the disclosure of which is incorporated by reference herein in its entirety as part of the present application.

### TECHNICAL FIELD

The present disclosure relates to the field of medical technology, and in particular to a method for positioning and generating titanium plate for internal fixation of jawbone, as well as a device, a medium, and a product.

### BACKGROUND

Titanium plates are important instruments in maxillofacial surgery for treating dentofacial development deformities and trauma, which are widely used in the treatment of both adolescent and adult patients and applicable to cases of jaw deformities caused by genetic factors, developmental abnormalities, or traumatic injuries. Titanium alloy material endows the titanium plate with the characteristics of light weight, high strength and corrosion resistance, with good biocompatibility, which can effectively reduce postoperative complications, ensure the stability of therapeutic effect, and have little impact on the comfort of the patient after surgery. At present, the mainly used type is a standard titanium plate, which needs to be manually bent by doctors for internal fixation during surgery. During treatment, the titanium plate is surgically fixed to the cortical parts of the maxilla and mandible to immobilize bone fractures and provide stress support.

With the advancement of orthognathic surgical techniques, the morphology of titanium plates has been continuously optimized. However, it is impossible to automatically generate a patient-specific titanium plate that is in closely fit with the bone surface, and the manual design operation is complex. Currently, standard titanium plate positioning and placement methods still have significant defects, including primarily relying on subjective judgment of the surgeons to identify the thickest part of a bone wall during surgery, followed by manual bending and attachment of the titanium plate of uniform specification and model to the bone surface. This process is specifically as follows: the surgeon first locates a region regarded as the thickest part of the bone surface on the jawbone of the patient based on experience, then manually bends the standard titanium plate to make the titanium plate fit the bone surface as much as possible, finally completing the fixation of the titanium plate. However, this method is not supported by precise quantitative evidence, which is highly prone to human errors, and is difficult to ensure that each screw hole is located at the thickest part of the bone surface, leading to great reduction of the matching accuracy between the titanium plate and the jawbone of the patient. In addition, the standard titanium plates have limited morphological options, which affects surgical accuracy and therapeutic outcomes, and prolongs the rehabilitation period of the patient.

### SUMMARY

An objective of the present disclosure is to provide a method for positioning and generating a titanium plate for internal fixation of jawbone, as well as a device, a medium, and a product, which can solve the problem of inaccurate generation and positioning of the titanium plate for internal fixation of jawbone caused by manual operation.

To achieve the objective described above, the present disclosure provides the following solutions.

In a first aspect, the present disclosure provides a method for positioning and generating a titanium plate for internal fixation of jawbone, including the following steps: performing Computed Tomography (CT) or Cone-beam Computed Tomography (CBCT) scanning on a bone surface of a patient to determine a bone surface digital model, wherein the bone surface digital model is stored in a form of mesh data, and the bone surface comprises the jawbone; performing surface smoothing processing on the mesh data to determine each mesh point corresponding to smoothed mesh data; determining a normal vector of each triangular patch based on an adjacent triangular patch formed by each mesh point; determining a bone surface thickness value corresponding to each mesh point according to the normal vector of each triangular patch; mapping the bone surface thickness value corresponding to each mesh point to the bone surface digital model to generate a thickness map; identifying a mesh region corresponding to a maximum bone surface thickness value based on the thickness map, and reversely mapping the mesh region to the bone surface digital model to determine a target triangular patch on the bone surface digital model corresponding to the mesh region; generating positioning of each titanium plate screw hole based on a normal vector of the target triangular patch and a predetermined range of thickness threshold; and generating the titanium plate for internal fixation of jawbone by using a bone surface mesh patch offset method according to the positioning of each titanium plate screw hole, wherein the bone surface mesh patch offset method comprises a uniform offset method.

In a second aspect, the present disclosure provides a computer device, including a memory, a processor, and a computer program stored on the memory and executable on the processor, wherein the processor is configured to execute the computer program to implement the method for positioning and generating a titanium plate for internal fixation of jawbone described above.

In a third aspect, the present disclosure provides a computer-readable storage medium, on which a computer program is stored, where the computer program, when executed by a processor, is configured to implement the method for positioning and generating a titanium plate for internal fixation of jawbone described above.

In a fourth aspect, the present disclosure provides a computer program product, including a computer program, where the computer program, when executed by the processor, is configured to implement the method for positioning and generating a titanium plate for internal fixation of jawbone described above.

According to specific embodiments of the present disclosure, the present disclosure has the following technical effects.

According to the present disclosure, the bone surface of the patient is subjected to CT or CBCT scanning to determine the bone surface digital model, mesh data corresponding to the bone surface digital model undergoes surface smoothing processing to determine each mesh point corresponding to the smoothed mesh data, thereby achieving automatic analysis of the mesh data. The normal vector of each triangular patch is determined based on the adjacent triangular patch formed by each mesh point, the bone surface thickness value corresponding to each mesh point is determined based on the normal vector of the triangular patch, and the bone surface thickness value corresponding to each mesh point is mapped to the bone surface digital model to generate the thickness map. Then, the mesh region corresponding to the maximum bone surface thickness value is automatically and accurately identified, then reverse mapping is performed to determine a corresponding target triangular patch on the bone surface digital model corresponding to the mesh region, and the position of each titanium plate screw hole is generated based on the normal vector of the target triangular patch and the predetermined thickness range, thereby achieving accurate positioning of the titanium plate. According to the present disclosure, the mesh region corresponding to the maximum bone surface thickness value is quickly and accurately identified, and then the corresponding target triangular patch on the bone surface digital model is intelligently identified to achieve the design of the titanium plate for internal fixation of the jawbone that accurately matches the bone surface morphology, avoid the interference of human factors, and improve the matching accuracy between the titanium plate for internal fixation of jawbone and the jawbone of the patient, thereby improving the success rate of the surgery and promoting patient rehabilitation better.

### BRIEF DESCRIPTION OF THE DRAWINGS

To describe the technical solutions in the embodiments of the present disclosure or in the prior art more clearly, the following briefly introduces the accompanying drawings required for describing the embodiments. Apparently, the accompanying drawings in the following description show merely some embodiments of the present disclosure, and a person of ordinary skill in the art may still derive other drawings from these accompanying drawings without creative efforts.

FIG. 1 is a diagram of a flow for generating and positioning of a Bone-anchored maxillary protraction (BAMP) plate according to an embodiment of the present disclosure;

FIG. 2 is a diagram showing feature points in a mesh region corresponding to a maximum bone surface thickness value identified based on a thickness map according to an embodiment of the present disclosure;

FIG. 3 is a diagram of a hollow cylinder generated according to an embodiment of the present disclosure;

FIG. 4 is a diagram of a titanium plate formed after bridge connection according to an embodiment of the present disclosure.

### DETAILED DESCRIPTION OF THE EMBODIMENTS

The following clearly and completely describes the technical solutions in the embodiments of the present disclosure with reference to the accompanying drawings in the embodiments of the present disclosure. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

To make the foregoing objectives, features and advantages of the present disclosure more apparent and easier to understand, the present disclosure will be further described in detail with the accompanying drawings and specific embodiments.

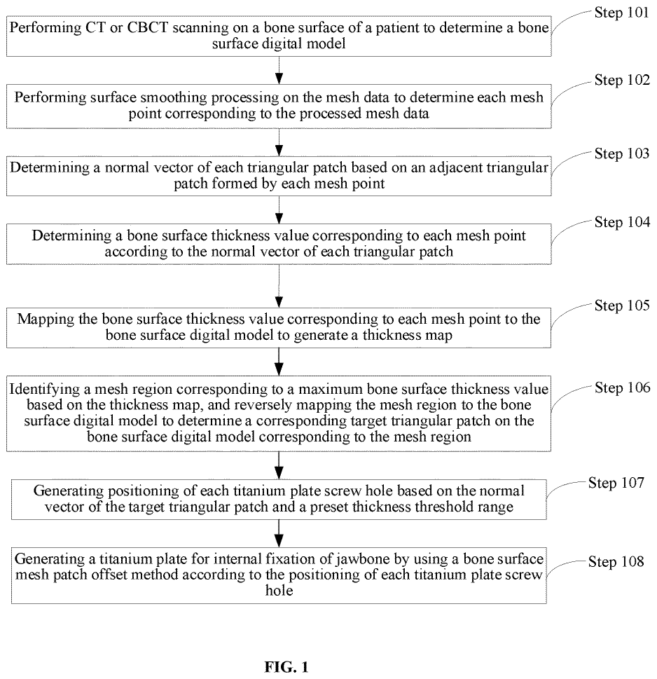

As shown in FIG. 1, the present disclosure provides a method for positioning and generating a titanium plate for internal fixation of a jawbone, including the following steps.

Step 101. A bone surface of a patient is subjected to CT or CBCT scanning to determine a bone surface digital model, where the bone surface digital model is stored in a form of mesh data, and the bone surface includes jawbone.

Step 102. The mesh data is subjected to surface smoothing processing to determine each mesh point corresponding to smoothed mesh data.

In some embodiments, step 102 specifically includes the following steps: for any mesh point and a predetermined range, a weight of each neighboring mesh point within the predetermined range is determined by using a Gaussian kernel function, where the Gaussian kernel function is determined according to a distance from each neighboring mesh point to the mesh point. The weight of each neighboring mesh point is normalized. Based on the normalized weight of each neighboring mesh point, the mesh point and each neighboring mesh point are subjected to weighted sum to determine an updated mesh point and each updated neighboring mesh point. Respective updated mesh points and respective updated neighboring mesh point serve as mesh points corresponding to the smoothed mesh data.

Step 103. A normal vector of each triangular patch is determined based on an adjacent triangular patch formed by each mesh point.

Step 104. A bone surface thickness value corresponding to each mesh point is determined according to the normal vector of each triangular patch.

In some embodiments, Step 104 specifically includes the following steps: for the normal vector of any triangular patch and any vertex of the triangular patch, a vertical distance from the vertex to the other side of the bone surface along a direction of the normal vector is determined, the vertical distance serves as a bone surface thickness value corresponding to the vertex, and the bone surface thickness value corresponding to each vertex serves as the bone surface thickness value corresponding to each mesh point.

In some embodiments, for the normal vector of any triangular patch and any vertex of the triangular patch, determining the vertical distance from the vertex to the other side of the bone surface along the direction of the normal vector specifically includes the following steps: the vertex is moved along the direction of the normal vector according to a predetermined magnification factor to form a search point. A nearest point of the search point is searched for, by using KDTree, to determine a distance between the search point and the nearest point, and the distance between the search point and the nearest point serves as the vertical distance from the vertex to the other side of the bone surface along the direction of the normal vector.

Step 105. The bone surface thickness value corresponding to each mesh point is mapped to the bone surface digital model to generate a thickness map.

In some embodiments, Step 105 specifically includes the following steps: based on the bone surface thickness value corresponding to each mesh point, the bone surface thickness value corresponding to each mesh point is mapped to the bone surface digital model, by using a Viridis color mapping method, to generate the thickness map.

Step 106. A mesh region corresponding to a maximum bone surface thickness value is identified based on the thickness map, and the mesh region is reversely mapped to the bone surface digital model to determine a corresponding target triangular patch on the bone surface digital model corresponding to the mesh region.

The mesh region corresponding to the maximum bone surface thickness value can be identified from the thickness map through a predetermined maximum bone surface thickness threshold.

Step 107. Positioning of each titanium plate screw hole is generated based on the normal vector of the target triangular patch and a predetermined range of thickness threshold.

Step 108. The titanium plate for internal fixation of jawbone is generated by using a bone surface mesh patch offset method according to the positioning of each titanium plate screw hole.

In some embodiments, Step 107 specifically includes the following steps: based on the normal vector of the target triangular patch, the predetermined number of hollow cylinders within a predetermined range of thickness threshold is generated from bottom to top at a predetermined spacing by using a constructive solid geometry algorithm; and the predetermined number of hollow cylinders are used as positions of various titanium plate screw holes.

During practical application, a thickest region on the bone surface is identified, and then four points are generated in the thickest region from top to bottom, where the fourth points are respectively generated on four triangular patches (the four points are generated randomly), a hollow cylinder with an axis parallel to the normal vector is generated in the direction according to the normal vector of each triangular patch, and the triangular patch is located at the center of the hollow cylinder.

In some embodiments, Step 108 specifically includes the following steps: based on the positioning of each titanium plate screw hole, titanium plate screw holes in bridge connection are generated between the positioning of titanium plate screw holes by using the bone surface mesh patch offset method; and the titanium plate for internal fixation of the jawbone is generated based on various titanium plate screw holes in bridge connection.

Specifically, in Step 107 and Step 108, according to the generated titanium plate screw holes, where a personalized titanium plate can be generated the screw holes in bridge connection are generated by using the bone surface mesh patch offset method between the positioning of screw holes, can be generated a personalized titanium plate. As mesh penetration exists in a constructed geometric entity relative to a jawbone model, the personalized titanium plate needs to undergo draft optimization from the jawbone to generate an intelligent titanium plate for internal fixation of jawbone, and the intelligent titanium plate is then 3D-printed and used for precise surgical placement in the jawbone.

In practical application, the reconstruction and production of existing titanium plates are entirely dependent on manual operations performed by doctors or technicians. Manual identification of a thickest position of a bone wall lacks objective standards, and leading to significant differences in the judgments of different operators. Based on this, the hole and morphology design of the titanium plate also varies from person to person, and the operation process is cumbersome and easy to fatigue. These factors lead to inevitable human error, which eventually greatly reduces the matching accuracy of the titanium plate and the jawbone of the patient jaw, affects the accuracy of surgery and treatment effect, and prolongs the rehabilitation time of the time.

From identifying the thickest position of the bone wall to completing the production of the titanium plate, all links need manual participation. The manual operation process is complicated, including position searching, shape designing, titanium plate bending, and the like, leading to the consumption of a significant amount of time. During emergency clinical surgery, this low efficiency cannot provide an appropriate titanium plate in time, which may delay the optimal treatment opportunity of the patient and adversely affect the condition of the patient.

Manual design and production of the titanium plate require professional technicians, leading to high labor cost. In addition, the design and production also rely on some high-precision expensive devices. Furthermore, a longer production period increases the time cost. The superposition of these factors makes the production cost of titanium plate persistently high, which not only aggravates the operating burden of medical institutions, but also indirectly leads to the increase of treatment cost and economic pressure of the patient.

By introducing algorithms for automatic thickness detection of the digital model, thickness map generation, and automatic reverse modeling and generation of the titanium plate, the data of the bone surface digital model is automatically analyzed by using a computer program and a mathematical model. Through accurate algorithm calculation, the thickest position of the bone wall is automatically identified, and a titanium plate design that accurately matches the bone surface morphology is generated, so that the interference of human factors is avoided, the matching accuracy between the titanium plate and the jawbone of the patient is improved, and the success rate of surgery is improved, promoting the rehabilitation of the patient better.

Through the adoption of the automatic design and 3D printing technology, the whole process from obtaining the bone surface digital model to generating the titanium plate can be processed automatically and quickly. Compared with the conventional manual operation, the period from design to production is greatly shortened. In the case of emergency surgery, the required titanium plate for clinic can be rapidly provided to ensure that the patient can get timely and effective treatment. The automatic production reduces the dependence on a large number of professionals and reduces the labor cost. In addition, the demands on some expensive devices are reduced, the production period is shortened, and the time cost is reduced. These changes make the production of the titanium plate more economic and efficient and reduce the operating cost of medical institutions and the economic burden of the patient. According to the present disclosure, the titanium plate is automatically generated and positioned by the thickness map of the digital model, and the matching accuracy between the titanium plate and the jawbone of the patient is achieved, with specific process as follows.

First, some technical terms are explained as follows.

Bone surface digital model: the bone surface digital model is a three-dimensional bone surface model obtained by CT or CBCT scanning, which is presented in the form of mesh data (point cloud or triangular mesh) and can accurately record a geometric shape, position and other information of the bone surface, thereby providing a foundation for subsequent analysis and processing.

Surface smoothing: noise may be generated during scanning, resulting in irregular fluctuations on the surface of the mesh model. The surface smoothing is to process the mesh by applying a specific algorithm to eliminate these noises and make the surface of the model flatter, thereby ensuring the accuracy of thickness calculation. Common algorithms, such as Laplacian smoothing algorithm, can be used to achieve surface smoothing by adjusting a vertex position to approach an average position of its neighborhood vertices.

Normal vector: the normal vector refers to a vector perpendicular to a bone surface triangular mesh plane of the bone surface digital model, and reflects directional characteristics of the bone surface at this point. In the present disclosure, the normal vector is calculated by the cross product of vectors of adjacent triangular patches, and is used to determine the direction for thickness calculation.

Thickness map: the thickness map is a map formed by displaying a thickness value of each point of the bone surface on the bone surface digital model in a color mapping manner. Different colors represent different thicknesses, facilitating intuitive observation of the thickness distribution of the bone surface. For example, warm colors indicate regions with greater thickness, whereas cool colors indicate regions with smaller thickness.

Constructive Solid Geometry (CSG) algorithm: the algorithm is a solid modeling algorithm that combines simple geometric entities (such as cubes and spheres) through Boolean operations (union, intersection, difference, and the like) to construct a complex three-dimensional model, which is used in the present disclosure for designing the morphology of the titanium plate.

KDTree (K-Dimensional Tree): the KDTree is a data structure for storing and quickly retrieving K-dimensional spatial data. In the thickness calculation of the present disclosure, the data structure is used to quickly search for the nearest point in the direction of the normal vector, thereby improving calculation efficiency.

### Step One: a Bone Surface Digital Model is Obtained.

Specifically, CT or CBCT scanning is adopted, laser scanning, structured light scanning or CT/MRI (Magnetic Resonance Imaging) can be adopted to perform omnidirectional scanning on the bone surface (mainly jawbone) of the patient. The laser scanning refers to measuring a surface distance of an object with a laser beam to obtain high-accuracy three-dimensional data. The structured light scanning refers to reconstructing a shape of the object by projecting a specific pattern and analyzing an image. The CT/MRI can obtain internal structure information of bone tissue and provide data basis for subsequent processing. The model obtained by scanning is stored in the form of mesh data, such as point cloud or triangular mesh, which includes a detailed geometric shape, vertex coordinates, connection relationship, and other information of the bone surface.

CT scanning is the primary method used, where a scanning slice thickness is less than 1 mm, and a scanning range is a craniomaxillofacial region including the whole mandible and maxilla. The jawbone can be reconstructed automatically and effectively by CT scanning with small slice thickness, thereby avoiding the loss of bone thickness in reconstruction.

### Step 2. Mesh Data is Subjected to Surface Smoothing Processing.

Specifically, due to the influences of device accuracy and environmental factors in the scanning process, noise inevitably exists in the obtained mesh data, which may cause irregular tiny fluctuations on the surface of the model. If the thickness is calculated directly based on the model with the noise, the calculation result will be inaccurate. Therefore, the mesh needs to undergo surface smoothing processing. Common smoothing algorithms, such as a Laplacian smoothing algorithm and Taubin algorithm, may be adopted. The Laplacian smoothing algorithm involves calculating an average position of each vertex and its neighborhood vertices, moving the vertex to the average position by a certain proportion, thereby making the model surface smoother. The Taubin algorithm, on the basis of Laplacian smoothing, can balance the relationship between surface smoothing and model feature retention better by adjusting iterative parameters.

In this embodiment, Gaussian smoothing is used, which is also widely used in three-dimensional mesh processing. Smoothing is achieved by performing weighted average on each vertex of the triangular patch and its neighborhood, and a Gaussian kernel function is generally used to define the weight. The advantage of the Gaussian smoothing is that it can preserve important geometric features while denoising, especially for a smoother surface.

A key parameter of Gaussian smoothing is a standard deviation (σ, Sigma), which controls a width of a Gaussian kernel and then affects the intensity of smoothing. Specifically, a formula of the Gaussian function is as follows:

$$w ( i ) = exp ( - \frac{{( {d}_{i} )}^{2}}{( 2 {σ}^{2} )} )$$

where w(i) is a weight of an i^{-th} neighborhood vertex, di is a distance from the i^{-th} neighborhood vertex to a current vertex; and σ is a standard deviation of Gaussian distribution, which determines a weight of weight distribution, and then affects the smoothing effect.

1. Selection of σ: if a smaller standard deviation is selected, more details are preserved, which is suitable for an occasion where the shape is relatively complex and it is not desired to lose important features. For example, it is necessary to preserve more local detail and edge information in a jawbone mesh region of the bone surface digital model. Generally, it may be set to 0.1-0.2 times the mesh size.

2. Neighborhood size: the neighborhood range (that is, the number of points affecting smoothing) is usually selected within 3 times σ. If the neighborhood size is overly small, smoothing effect will be insignificant, and if the neighborhood size is excessively large, the model will be excessively smoothed, leading to the loss of details.

3. The number of iterations: the number of iterations of Gaussian smoothing (that is, the number of smoothing applications to be performed on the vertices of each triangular patch) will also affect the final result. In general condition, three iterations can achieve a good smoothing effect. If the noise is prominent or intensive denoising is required, the number of iterations will be appropriately increased.

### Step 3. Thickness Calculation

Specifically, normal vector calculation involves calculating a surface normal vector for each mesh point by the cross product of vectors of geometric information of its adjacent triangular patches.

A thickness calculation method includes that the thickness of the bone surface is defined as the vertical distance from a specific point of the triangular patch to the other side of the bone surface along the direction of normal vector of the triangular patch. To calculate the thickness, after the normal vector is calculated, a search point is extended in the direction of the normal vector. The normal vector magnification is set (the current value is 10), which can not only ensure the accuracy of bone thickness measurement of an anterior wall of the maxillary sinus, but also ensure that the bone thickness of a posterior part not belonging to the maxilla will not be included when the bone thickness near the zygomatic alveolar ridge is measured, thereby accurately generating a bone thickness map. The KDTree data structure is used to find the nearest point (R) of the search point (Q) in the model, and a distance from point (P) to point (R) is the bone surface thickness at this point. It is crucial to choose the appropriate normal vector magnification. If the magnification is overly small, the search point may not be able to cross the bone surface to find the nearest point on the other side, which makes a thickness calculation result smaller. If the multiple is excessively large, too many irrelevant points will be introduced, which affects the calculation efficiency and accuracy.

Step Four. Identification of Thickest Part

Generation of thickness map: a calculated thickness value of each point is mapped to a 3D model to generate the thickness map. When the thickness map is generated, it is crucial to select an appropriate color mapping scheme. The present disclosure employs a Viridis color mapping scheme, and an important feature of Viridis color mapping design is that it has good perceptual uniformity. In other words, the change in a data value is linear visually, that is, the transition of color is smooth and uniform. This is crucial to convey small differences in data, especially in scientific data visualization that requires high accuracy. In addition, Viridis employs a color spectrum from purple to yellow to ensure that all color changes are recognizable to different backgrounds and color-blind users. The visual deviation that may be introduced by many conventional color maps (such as jet) can be avoided, which often make the difference between high and low values difficult to identify, and map a thickness value from a minimum value to a maximum value as a gradient color from a cool color (such as blue) to a warm color (such as red). In this case, on the thickness map, the thickness difference at different positions of the bone surface can be intuitively distinguished through the change of color, facilitating subsequent analysis and processing.

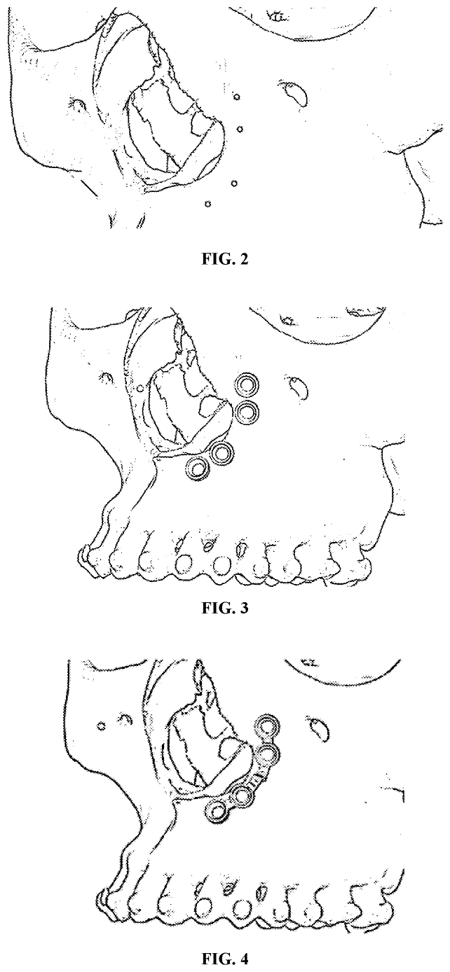

Detection of maximum thickness: by traversing all the thickness values in the thickness map, the mesh region corresponding to the maximum thickness value is found, which is the thickest part of the bone surface, and four feature points are selected from top to bottom in this part, as shown in FIG. 2. In actual programming implementation, a cycle structure can be used to compare the data of the thickness map point by point, thereby recording the maximum value and the corresponding position information. To improve detection efficiency, some optimization algorithms may also be adopted, such as a divide-and-conquer method, which divides the thickness map into multiple sub-regions, searches for the maximum values in sub-regions separately, and finally merges the results to reduce the number of comparisons and improve the search speed.

Step Five. Automatic Generation of Titanium Plate

As shown in FIG. 5, after the thickest part of the bone surface is identified by using the thickness map, the four points at the thickest part marked on the bone surface are reversely mapped as titanium nail implant sites, and a modeling algorithm (such as CSG algorithm) is used for the design of the morphology of the titanium plate to automatically generate four hollow cylinders. For example, the cylinder has a height of 0.8 mm and an outer diameter of 5.5 mm, and a hollow inner diameter for placing the titanium nail is 3.0 mm. A bone surface offset cube is generated among the four hollow cylinders to connect four hollow cylinders, the cube has a height of 0.8 mm and a width of 3 mm, the length is automatically generated according to a spacing between the cylinders, thereby achieving automatic design.

With reference to FIG. 3 and FIG. 4, the constructive solid geometry (CSG) technology is configured to determine some basic geometric shapes at first. For example, the hollow cylinder is used to represent a hole of the titanium plate, and a cuboid is used to represent a main body part of the titanium plate. Afterwards, by Boolean operation (such as by using the union operation, the cylinder and the cuboid are combined to form a basic shape of the titanium plate with holes; an edge of the titanium plate can be rounded by intersection operation to make the titanium plate fit the bone surface better, thereby avoiding damaging the surrounding tissues; the difference operation can remove unnecessary parts and optimize the titanium plate structure) to construct the shape of the titanium plate that meets the clinical needs. During design, it is also necessary to consider factors such as the mechanical properties of the titanium plate and the degree of fit with the bone surface, and the thickness of the titanium plate and the distribution of holes (that is, hollow cylinders) need to be adjusted according to the curvature and stress of the bone surface to ensure that the titanium plate can be stably fixed on the bone surface while providing sufficient traction. Holes are positions corresponding to the four points at the thickest part.

Fabrication and printing: according to the designed titanium plate model, 3D printing technology, such as selective laser melting (SLM), is used for automatic generation of the titanium plate. According to the 3D printing technology, materials can be stacked layer by layer according to the digital model to accurately fabricate the required titanium plate. During printing, it is crucial to select an appropriate printing material (such as a specific type of titanium alloy, which has good biocompatibility, strength and corrosion resistance) and printing parameters (such as a printing layer thickness, a printing speed, and a printing temperature). The thickness of the printing layer has an influence on the surface quality and accuracy of the titanium plate. During a smaller layer thickness, a smoother surface can be obtained, but the printing time may be increases. Printing speed has an influence on the production efficiency, but too fast speed may lead to the decline of the printing quality, resulting in some problems such as poor interlayer bonding. The printing temperature has an influence on the fluidity and curing effect of the material. An appropriate temperature can ensure the uniform accumulation of the materials and improve the mechanical properties of the titanium plate.

In an example embodiment, the present disclosure further provides a computer device including a memory and a processor. A computer program is stored in the memory, and the processor, when executing the computer program, can implement the foregoing method.

In an example embodiment, the present disclosure further provides a computer-readable storage medium, on which a computer program is stored. The computer program, when executed by a processor, is configured to implement the foregoing method.

In an example embodiment, a computer program product is provided, which includes a computer program. The computer program, when executed by a processor, is configured to implement the foregoing method.

It should be noted that both user information (including, but not limited to, user equipment information, personal user information, and the like) and data (including, but not limited to, data for analysis, stored data, displayed data, and the like) involved in the present disclosure are information and data authorized by users or fully authorized by all parties, and the collection, use and processing of relevant data need to comply with relevant regulations.

Those skilled in the art may understand that all or part of the processes in the method for implementing the foregoing embodiments can be completed by instructing related hardware through a computer program. The computer program can be stored in a nonvolatile computer-readable storage medium, and when executed, the computer program may include the processes of the foregoing embodiments. Any reference to memory, database or other media used in the embodiments provided in the present disclosure may include at least one of a nonvolatile memory and a volatile memory. The nonvolatile memory may include a read memory (ROM), a magnetic tape, a floppy disk, a flash memory, an optical memory, a high density embedded nonvolatile memory, a resistive random access memory (ReRAM), a magnetoresistive random access memory (MRAM), a ferroelectric random access memory (FRAM), a phase change memory (PCM), a graphene memory, and the like. The volatile memory may include a random access memory (RAM), or an external cache memory. By way of illustration than limitation, RAM may be in various forms, such as a static random access memory (SRAM) or a dynamic random access memory (DRAM).

A database involved in each embodiment provided by the present disclosure may include at least one of a relational database and a non-relational database. The non-relational database may include, but is not limited to, a distributed database based on a block chain. The processor involved in the embodiments provided by the present disclosure may be a general processor, a central processing unit, a graphics processing unit, a digital signal processor, programmable logics, data processing logics based on quantum computing, and the like, without limitation.

Various technical features of the foregoing embodiments may be combined arbitrarily. For the sake of brevity, all possible combinations of the technical features in the above embodiments are not described. However, the combinations of these technical features should be considered to be within the scope of this specification as long as there is no contradiction therebetween.

Several examples are used for illustration of the principles and implementations of the present disclosure. The description of the embodiments is merely used to help illustrate the method and its core principles of the present disclosure. In addition, a person of ordinary skill in the art can make various modifications in terms of specific implementations and scope of application in accordance with the teachings of the present disclosure. In conclusion, the content of this specification shall not be construed as a limitation to the present disclosure.

What is claimed is:

1. A method for positioning and generating a titanium plate for internal fixation of a jawbone, comprising:
performing Computed Tomography (CT) or Cone-beam Computed Tomography (CBCT) scanning on a bone surface of a patient to determine a bone surface digital model, wherein the bone surface digital model is stored in a form of mesh data, and the bone surface comprises the jawbone;
performing surface smoothing processing on the mesh data to determine each mesh point corresponding to smoothed mesh data;
determining a normal vector of each triangular patch based on an adjacent triangular patch formed by each mesh point;
determining a bone surface thickness value corresponding to each mesh point according to the normal vector of each triangular patch comprising:
for a normal vector of any triangular patch and any vertex of the triangular patch, determining a vertical distance from the vertex to an other side of the bone surface along a direction of the normal vector, and serving the vertical distance as a bone surface thickness value corresponding to the vertex; and
serving the bone surface thickness value corresponding to each vertex as the bone surface thickness value corresponding to each mesh point;
mapping the bone surface thickness value corresponding to each mesh point to the bone surface digital model to generate a thickness map;
identifying a mesh region corresponding to a maximum bone surface thickness value based on the thickness map, and reversely mapping the mesh region to the bone surface digital model to determine a target triangular patch on the bone surface digital model corresponding to the mesh region;
generating positioning of each titanium plate screw hole based on a normal vector of the target triangular patch and a predetermined range of thickness threshold comprising:
based on the normal vector of the target triangular patch, using a constructive solid geometry algorithm to generate a predetermined number of hollow cylinders within the predetermined range of thickness threshold from bottom to top at a predetermined spacing, which comprising:
identifying a thickest region on the bone surface; generating four points in the thickest region from top to bottom at the predetermined spacing, wherein the four points are respectively generated on the target triangular patch; and generating a hollow cylinder with an axis parallel to the normal vector in the direction according to the normal vector of the target triangular patch, wherein the predetermined number is four;
serving the predetermined number of hollow cylinders as positioning of titanium plate screw holes; and
generating the titanium plate for internal fixation of jawbone by using a bone surface mesh patch offset method according to the positioning of each titanium plate screw hole, wherein the bone surface mesh patch offset method comprises a uniform offset method.

2. The method for positioning and generating the titanium plate for internal fixation of the jawbone according to claim 1, wherein performing the surface smoothing processing on the mesh data to determine each mesh point corresponding to the smoothed mesh data comprises:
for any mesh point and the predetermined range, determining a weight of each neighboring mesh point within the predetermined range by using a Gaussian kernel function, wherein the Gaussian kernel function is determined according to a distance between each neighboring mesh point and the mesh point;
normalizing the weight of each neighboring mesh point;
performing weighted sum on the mesh point and each neighboring mesh point based on the weight of each neighboring mesh point normalized to determine an updated mesh point and each updated neighboring mesh point; and
serving respective updated mesh points and respective updated neighboring mesh points as each mesh point corresponding to the smoothed mesh data.

3. The method for positioning and generating the titanium plate for internal fixation of the jawbone according to claim 1, wherein generating the titanium plate for internal fixation of the jawbone by using the bone surface mesh patch offset method according to the positioning of each titanium plate screw hole comprises:
based on the positioning of each titanium plate screw hole, generating titanium plate screw holes in bridge connection between the positioning of titanium plate screw holes by using the bone surface mesh patch offset method; and
generating the titanium plate for internal fixation of the jawbone based on the titanium plate screw holes in the bridge connection.

4. The method for positioning and generating the titanium plate for internal fixation of the jawbone according to claim 1, wherein mapping the bone surface thickness value corresponding to each mesh point to the bone surface digital model to generate the thickness map comprises:
based on the bone surface thickness value corresponding to each mesh point, mapping, by using a Viridis color mapping method, the bone surface thickness value corresponding to each mesh point to the bone surface digital model to generate the thickness map.

5. The method for positioning and generating the titanium plate for internal fixation of the jawbone according to claim 1, wherein for the normal vector of any triangular patch and any vertex of the triangular patch, determining the vertical distance from the vertex to the other side of the bone surface along the direction of the normal vector, comprises:
moving the vertex along the direction of the normal vector according to a predetermined magnification factor to form a search point; and
searching for a nearest point to the search point by using K-Dimensional Tree (KDTree) to determine a distance between the search point and the nearest point, and serving the distance between the search point and the nearest point as the vertical distance from the vertex to the other side of the bone surface along the direction of the normal vector.

6. A computer device, comprising a memory, a processor, and a computer program stored on the memory and executable on the processor, wherein the processor is configured to execute the computer program to implement the method for positioning and generating the titanium plate for internal fixation of the jawbone according to claim 1.

7. The computer device according to claim 6, wherein performing the surface smoothing processing on the mesh data to determine each mesh point corresponding to the smoothed mesh data comprises:
for any mesh point and the predetermined range, determining a weight of each neighboring mesh point within the predetermined range by using a Gaussian kernel function, wherein the Gaussian kernel function is determined according to a distance between each neighboring mesh point and the mesh point;
normalizing the weight of each neighboring mesh point;
performing weighted sum on the mesh point and each neighboring mesh point based on the weight of each neighboring mesh point normalized to determine an updated mesh point and each updated neighboring mesh point; and
serving respective updated mesh points and respective updated neighboring mesh points as each mesh point corresponding to the smoothed mesh data.

8. The computer device according to claim 6, wherein generating the titanium plate for internal fixation of the jawbone by using the bone surface mesh patch offset method according to the positioning of each titanium plate screw hole comprises:
based on the positioning of each titanium plate screw hole, generating titanium plate screw holes in bridge connection between the positioning of titanium plate screw holes by using the bone surface mesh patch offset method; and
generating the titanium plate for internal fixation of the jawbone based on the titanium plate screw holes in the bridge connection.

9. The computer device according to claim 6, wherein mapping the bone surface thickness value corresponding to each mesh point to the bone surface digital model to generate the thickness map comprises:
based on the bone surface thickness value corresponding to each mesh point, mapping, by using a Viridis color mapping method, the bone surface thickness value corresponding to each mesh point to the bone surface digital model to generate the thickness map.

10. The computer device according to claim 6, wherein for the normal vector of any triangular patch and any vertex of the triangular patch, determining the vertical distance from the vertex to the other side of the bone surface along the direction of the normal vector, comprises:
moving the vertex along the direction of the normal vector according to a predetermined magnification factor to form a search point; and
searching for a nearest point to the search point by using K-Dimensional Tree (KDTree) to determine a distance between the search point and the nearest point, and serving the distance between the search point and the nearest point as the vertical distance from the vertex to the other side of the bone surface along the direction of the normal vector.

11. A computer-readable storage medium, on which a computer program is stored, wherein the computer program, when executed by a processor, is configured to implement the method for positioning and generating the titanium plate for internal fixation of the jawbone according to claim 1.
